(19) United States
(12) Patent Application Publication
(10) Pub. No.: US 2007/0233073 A1
(43) Pub. Date: Oct. 4, 2007

(54) Spinal rod characterized by a time-varying stiffness

(75) Inventors: Paul Wisnewski, Maple Grove, MN (US); Joseph Lessar, Coon Rapids, MN (US); Dennis J. Buchanan, Loveland, OH (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/366,643

(22) Filed: Mar. 2, 2006

(51) Int. Cl. A61F 2/30

(52) U.S. Cl. 606/61

(57) ABSTRACT

A spinal rod characterized by a time-varying stiffness. The rod comprises a first member and at least one second member that is mechanically coupled to the first member through a time-varying interface. The interface features a binding mechanism that degrades after surgical installation. For instance, the interface may be bioabsorbable and dissolve upon exposure to bodily fluids. In another instance, the second member may be comprised of a bioabsorbable material. In another embodiment, the interface may fail under cyclic loading. In another embodiment, degradation of the bioabsorbable material may be inhibited through the application of a current source. The second member may be disposed within the first member. Alternatively, the first member and the second member may be disposed aside one another. The first member and the second member may be substantially similar in shape. One or more bioabsorbable caps may be used to at least temporarily seal the second member from bodily fluids once the spinal rod is installed.

### BACKGROUND

Spinal fusion is a surgical technique used to immobilize two or more vertebrae, often to eliminate pain caused by motion of the vertebrae. Conditions for which spinal fusion may be performed include degenerative disc disease, vertebral fractures, scoliosis, or other conditions that cause instability of the spine. One type of spinal fusion fixes the vertebrae in place with hardware such as hooks or pedicle screws attached to rods on one or each lateral side of the vertebrae. Often, the spinal fusion further contemplates a bone graft between the transverse processes or other vertebral protrusions. The bone graft may rely on supplementary bone tissue and bone growth stimulators in conjunction with the body's natural bone growth processes to literally fuse vertebral bodies to one another.

After a spine fusion surgery, it may take months for the fusion to successfully set up and achieve its initial maturity. During these first months, it is desirable to avoid loading that may place the bone graft at risk. Thus, during this initial period, the implanted rods should bear most if not all of the induced loads. The bone will continue to fuse and evolve over a period of months, if not years. Once established, the fused region should be robust enough to sustain normal spinal loads.

The bone growth process may be promoted, and the fused region may strengthen, if the fused region is subjected to increasing loads over time. Conventional spinal implants often use rigid or semi-rigid rods having a stiffness that does not change over time. Thus, the amount of loading that is carried by the implanted rods also does not vary with time.

### SUMMARY

Embodiments of the present application are directed to a spinal rod characterized by a time-varying stiffness. In certain embodiments, the rod includes a first member that is coupled to a second member to create a rod having a first rod stiffness. For instance, this first rod stiffness may reflect the stiffness of the rod prior to and immediately following surgical installation. This rod stiffness changes to a second rod stiffness after surgical installation. This may be implemented through a time-varying interface between the first and second members that degrades after surgical installation. In one embodiment, the rod may include a bioabsorbable or biodegradable second member whose cross sectional area or bonding interface or joining mechanism changes after exposure to bodily fluids. In other embodiments, the time varying interface may include a bioabsorbable or biodegradable adhesive between the first member and the second member.

### BRIEF DESCRIPTION OF THE DRAWINGS

FIG. 1 is a perspective view of first and second assemblies comprising fixation rods attached to vertebral members according to one or more embodiments;

FIG. 2 is a partial view of a spinal rod according to one or more embodiments;

FIG. 3 is a cross section view of a spinal rod according to one embodiment;

FIG. 4 is a cross section view of a spinal rod according to one embodiment;

FIG. 5 is a cross section view of a spinal rod according to one embodiment;

FIG. 6 is a cross section view of a spinal rod according to one embodiment;

FIG. 7 is a cross section view of a spinal rod according to one embodiment;

FIG. 8 is a cross section view of a spinal rod according to one embodiment;

FIG. 9 is a cross section view of a spinal rod according to one embodiment;

FIG. 10 is a cross section view of a spinal rod according to one embodiment;

FIG. 11 is a cross section view of a spinal rod according to one embodiment;

FIG. 12 is a longitudinal section view of a spinal rod according to one embodiment;

FIG. 13 is a longitudinal section view of a spinal rod according to one embodiment;

FIG. 14 is a longitudinal section view of a spinal rod according to one embodiment;

FIG. 15 is a side view of a spinal rod according to one embodiment;

FIG. 16 is a cross section view of a spinal rod according to one embodiment;

FIG. 17 is a longitudinal section view of a spinal rod according to one embodiment;

FIG. 18 is a cross section view of a spinal rod coupled to a current source according to one embodiment;

FIG. 19 is a cross section view of a spinal rod coupled to a current source according to one embodiment; and

FIG. 20 is a cross section view of a spinal rod coupled to a current source according to one embodiment.

### DETAILED DESCRIPTION

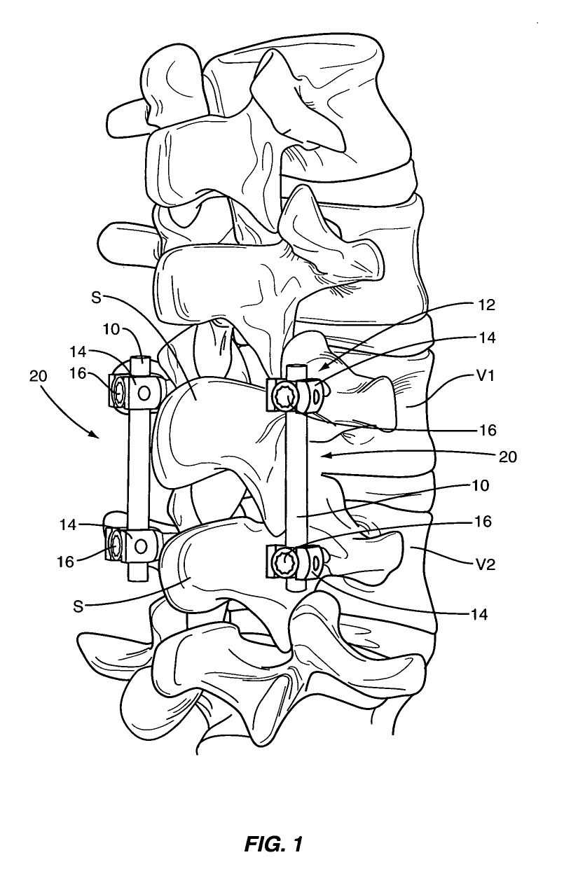

The various embodiments disclosed herein are directed to spinal rods that are characterized by a stiffness and load sharing capacity that change over time. Various embodiments of a spinal rod may be implemented in a spinal rod assembly of the type indicated generally by the numeral 20 in FIG. 1. FIG. 1 shows a perspective view of first and second spinal rod assemblies 20 in which spinal rods 10 are attached to vertebral members V1 and V2. In the example assembly 20 shown, the rods 10 are positioned at a posterior side of the spine, on opposite sides of the spinous processes S. Spinal rods 10 may be attached to a spine at other locations, including lateral and anterior locations. Spinal rods 10 may also be attached at various sections of the spine, including the base of the skull and to vertebrae in the cervical, thoracic, lumbar, and sacral regions. Thus, the illustration in FIG. 1 is provided merely as a representative example of one application of a spinal rod 10.

In the exemplary assembly 20, the spinal rods 10 are secured to vertebral members V1, V2 by pedicle assemblies 12 comprising a pedicle screw 14 and a retaining cap 16. The outer surface of spinal rod 10 is grasped, clamped, or otherwise secured between the pedicle screw 14 and retaining cap 16. Other mechanisms for securing spinal rods 10 to vertebral members V1, V2 include hooks, cables, and other such devices. Further, examples of other types of retaining hardware include threaded caps, screws, and pins. Spinal rods 10 are also attached to plates in other configurations. Thus, the exemplary assemblies 20 shown in FIG. 1 are merely representative of one type of attachment mechanism.

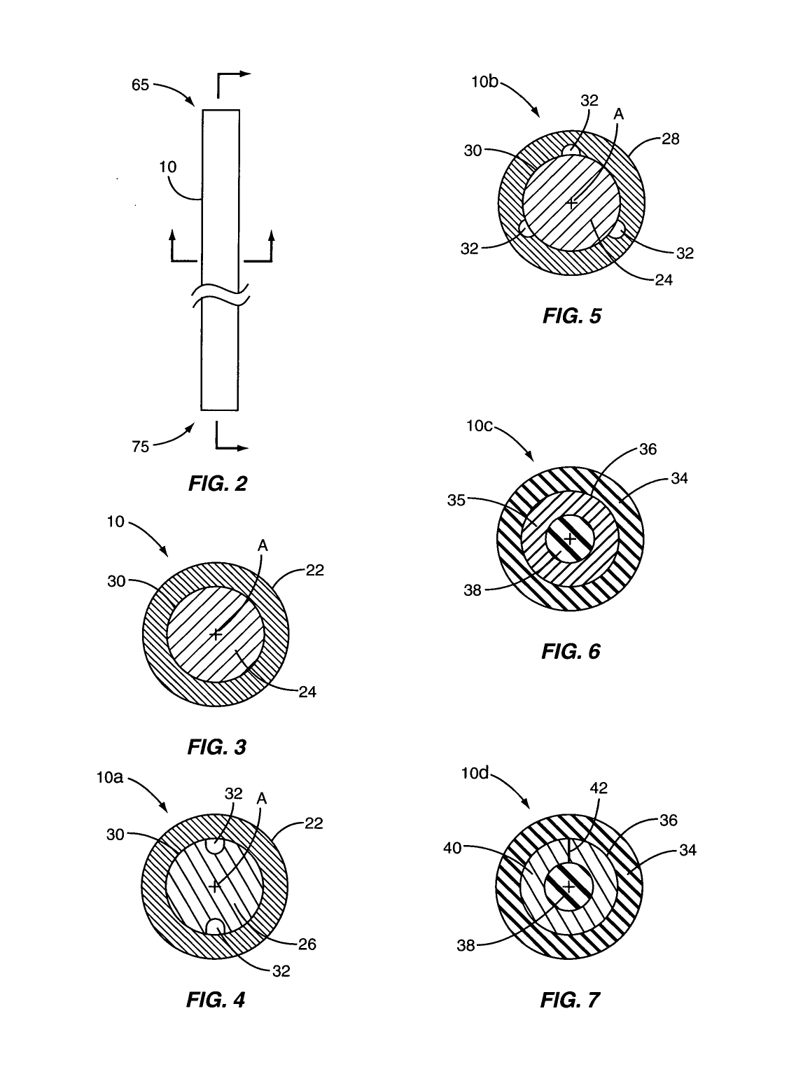

FIG. 2 shows a segment of a spinal rod 10 of the type used in the exemplary assembly 20 in FIG. 1. Other Figures described below show various embodiments of a spinal rod 10 characterized by different cross sections taken through the section lines illustrated in FIG. 2.

For instance, FIG. 3 shows one example cross section of the spinal rod 10. In this embodiment, the spinal rod 10 is comprised of a first member 22 encircling a second member 24. The first member 22 and second member 24 may be comprised of a biocompatible material. Suitable examples may include metals such as titanium or stainless steel, shape memory alloys such as nitinol, composite materials such as carbon fiber, and other resin materials known in the art. The second member 24 is comprised of a biocompatible, bioabsorbable or biodegradable material approved for medical applications. The term “bioabsorbable” generally refers to materials which facilitate and exhibit biologic elimination and degradation by the metabolism. Currently materials of this type, which are approved for medical use, include those materials known as PLA, PGA and PLGA. Examples of these materials include polymers or copolymers of glycolide, lactide, troxanone, trimethylene carbonates, lactones and the like.

The bioabsorbable or biodegradable material may be a metal as well. Corrosion is essentially the degradation of a metal by chemical attack. Thus, a similar result may be obtained through the use of bioabsorbable or biodegradable metals as with the exemplary bioabsorbable materials described above.

In one embodiment, the first member 22 and the second member 24 are bonded together at interface 30 with a bioabsorbable adhesive. In other embodiments, the bioabsorbable second member 24 is allowed to set and solidify within the first member 22, thus forming a bioabsorbable bond to the first member 22. In the present example, the interface 30 is substantially cylindrical. Initially, the interface 30 represents a secure coupling of the first member 22 and the second member 24. Thus, axial, flexural, and torsional stresses imparted on the rod 10 may be distributed among the first member 22 and second member 24. However, since the second member 24 in the present embodiment is bioabsorbable, the second member 24 will dissolve over time. Consequently, the axial, flexural, and torsional stiffness of the spinal rod 10 will change over time. This is due, in part, to the gradual change in cross sectional area, moments of inertia, and section modulus.

In certain embodiments, it is not necessary that the second member 24 completely degrade to achieve the desired change in stiffness. The stiffness of some bioabsorbable materials will change as they absorb fluid in-vivo. Thus, even where the first member 22 and the second member 24 remain coupled, the overall stiffness of the rod 10 may change as the stiffness of the second member 24 changes.

In the embodiment shown in FIG. 3, it may be the case that the bioabsorbable second member 24 will dissolve from the inside out, beginning at or near the longitudinal axis labeled A and progressing towards the interface 30. A variation, illustrated as spinal rod 10a in FIG. 4, may provide for a modified rate of decay. In this embodiment, the first member 22 is substantially similar to the embodiment shown in FIG. 3. A second member 26 is bioabsorbable similar to second member 24 except for the addition of one or more notches 32 disposed about the perimeter of the second member 26 near the interface 30. The notches 32 allow fluid infiltration through the entire rod 10a. This may accelerate decoupling of the first member 22 and second member 26 along the length of the rod 10a. The notches 32 may be cut parallel to axis A, cut in a spiral pattern about axis A, or a variety of other configurations.

Using a similar approach, the embodiment shown in FIG. 5 provides a series of notches 32 cut into first member 28. The second member 24 is substantially similar to the embodiment shown in FIG. 3. The first member 28 is similar to first member 22 except for the addition of one or more notches 32 disposed about the inside surface of the first member 28 near the interface 30. As above, the notches 32 allow fluid infiltration through the entire rod 10b and may accelerate decoupling of the first member 28 and second member 24 along the length of the rod 10b. Similarly, the notches 32 may be cut parallel to axis A, cut in a spiral pattern about axis A, and other configurations.

In an alternative embodiment shown in FIG. 6, the rod 10c is comprised of a first member 34, a second member 35, and a third member 38. In this embodiment, the first member 34 and second member 35 form concentric rings around the third member 38. In one embodiment, the third member 38 is fabricated using a bioabsorbable material while the first member 34 and second member 35 are fabricated from biocompatible materials that are not bioabsorbable. However, the interface 36 between the first member 34 and second member 35 is a bioabsorbable bond that dissolves over time similar to the entire third member 38. Thus, the present embodiment of the spinal rod 10c offers two modes of time-varying stiffness. The first contemplates a dissolving member 38 while the second contemplates a dissolving interface 36.

In one embodiment, the bioabsorbable material of third member 38 is chosen to have a faster rate of decay than that used in bonding the first and second members 34, 35 at interface 36. Initially, the stiffness of rod 10c is provided by a combination of the first, second, and third members 34, 35, 38. As the third member dissolves, a substantial majority of the stiffness in the rod 10c may be provided by the outer members 34, 35. However, the decay of the bond at interface 36 produces a second time-varying stiffness that ultimately results in the first member 34 solely contributing to the axial, flexural, and torsional stiffness of the rod 10c.

In an alternative embodiment shown in FIG. 7, the rod 10d is comprised of three members 34, 40, and 38. The structure of rod 10d is similar to the embodiment of rod 10c shown in FIG. 6. However, rod 10d is tuned to a different stiffness through the inclusion of a slotted second member 40. The slot 42 in second member 40 decreases the overall stiffness of the second member as compared to a similarly constructed second member 35 (FIG. 6). Initially, the slot 42 may not significantly decrease the overall axial, flexural, and torsional stiffness of rod 10d. However, once the third member 38 dissolves by a sufficient amount, the decreased stiffness in second member 40 due to slot 42 may contribute to an overall reduction in stiffness as compared to the embodiment of rod 10c shown in FIG. 6 for at least the period of time before the bond at interface 36 dissolves.

In an alternative embodiment shown in FIG. 8, the rod 10e is comprised of a first member 22 similar to FIG. 3. A plurality of second members 44 are disposed on the inside of the first member 22. In one embodiment, the second members 44 are bioabsorbable. In one embodiment, the second members 44 are bonded to one another and to the first member 22. In one embodiment, the second members 44 have a substantially cylindrical cross section. As shown, one or more open channels 46 exist between adjacent second members 44 and between the second members 44 and the first member 22. The channels 46 allow fluid infiltration through the entire rod 10e, which may accelerate decoupling of the first member 22 and second members 44 along the length of the rod 10e.

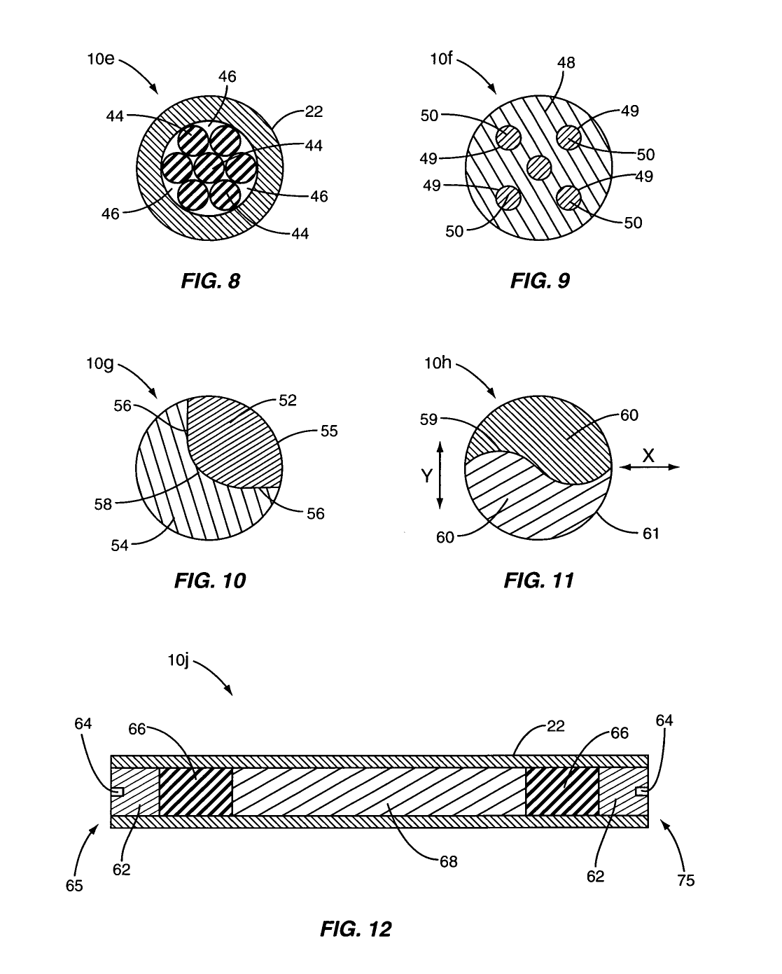

In an alternative embodiment shown in FIG. 9, the rod 10f is comprised of a first member 48 and a plurality of second members 50. The plurality of second members 50 are dispersed about the interior of the first member 48 within individual apertures formed by surfaces 49. In one embodiment, the second members 50 are bioabsorbable. Consequently, once the second members 50 dissolve, the first member 48 remains with a porous cross section having a different axial, flexural, and torsional stiffness as compared to when the rod 10f was initially installed.

FIG. 10 shows an alternative embodiment of rod 10g comprised of a first member 52 and a second member 54. In contrast with previous embodiments, rod 10g is not comprised of a hollow first member. Instead, the first and second members 52, 54 have complementary cross sections that, taken together, form a substantially circular outer perimeter 55. In one embodiment, the first and second members 52, 54 are bonded to one another. As with other embodiments, the bond at this interface may be bioabsorbable so that the two members 52, 54 separate from one another over time. The interface between the two members 52, 54 comprises a pair of slip planes 56 and a curved arc 58 therebetween. The slip planes 56 may increase flexural stiffness in a direction parallel to the plane 56. Once the bond at the interface dissolves, the slip planes serve to allow sliding motion at the interface, effectively reducing the stiffness of the combined structure having the circular cross section. Thus, the rod 10g may be inserted with the slip planes 56 oriented in desired directions to accommodate or inhibit certain anatomical motions.

FIG. 11 presents an alternative embodiment of rod 10h that is comprised of substantially similar first and second members 60. These members 60 have complementary cross sections that form a substantially circular outer perimeter 61 once assembled. In one embodiment, these members 60 are bonded to one another using a bioabsorbable adhesive so that the two members 60 separate from one another over time. Even after the bond layer at interface 59 disintegrates, the rod 10h may have greater bending flexibility (i.e., lower stiffness) in the direction of arrow Y than in the direction of arrow X. Thus, the rod 10h may be oriented in the patient to provide greater or lesser flexural stiffness in desired directions.

The embodiments described above have contemplated different cross sections and have not necessarily provided for varying rod construction in an axial direction. However, certain embodiments of the spinal rod 10 may have different constructions along its length to further tune its time-varying axial, flexural, and torsional stiffness. For instance, the embodiment shown in FIG. 12 shows a longitudinal cross section of an exemplary spinal rod 10j. In this embodiment, the rod 10j includes a first member 22 that is similar to embodiments shown in FIGS. 3, 4 and 8. A second member 68 is disposed interior to the first member 22. The second member 68 may be bioabsorbable and may be bonded to the first member 22 using a bioabsorbable adhesive.

Plugs 62 are inserted into first 65 and second 75 ends of the rod 10j. The plugs 62 may have a driving feature 64 (e.g., slot, hex, star, cross) that allows the plug 62 to be turned, twisted, pushed, or otherwise inserted into the ends of the rod 10j. In one embodiment, the exemplary plugs 62 are bioabsorbable and dissolve to expose a second series of plugs 66. These plugs 66 may also be bioabsorbable. Accordingly, the plugs 62, plugs 66, and second member 68 all may begin to dissolve at different points in time depending on when each is exposed to bodily fluids. Thus, as many or as few plugs 62, 66 may be used to tune the rate at which the axial, flexural, and torsional stiffness of the rod 10j varies.

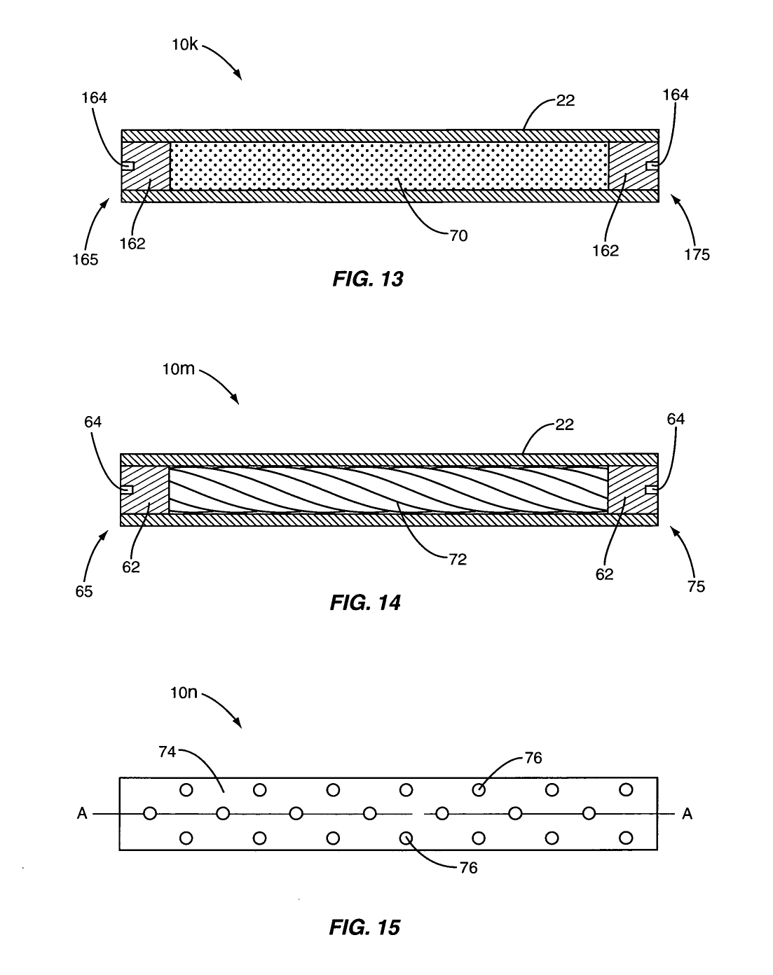

One embodiment of a rod 10k illustrated in FIG. 13 does not contemplate any bioabsorbable materials. Instead, a first member 22 that is similar to the embodiments shown in FIGS. 3, 4, 8, and 12 is capped at first 165 and second 175 ends by permanent plugs 162. The plugs 162 may have a driving feature 164 (e.g., slot, hex, star, cross) that allows the plug 162 to be turned, twisted, pushed, or otherwise inserted into the ends of the rod 10k. A powder metal 70 is disposed within the interior of the rod 10k. In one embodiment, the powder metal 70 may be comprised of particles having a size within a range between about 10 and 100 microns. Notably, since the inner cavity of rod 10k is substantially filled with the powder metal 70, the rod 10k may be clamped and bent to a desired installation shape without kinking the hollow first member 22.

During fabrication, the powder metal 70 may be compressed and lightly sintered. Sintering is a process used in powder metallurgy in which compressed metal particles are heated and fused. In the present embodiment, the sintering process does not necessarily heat the particles to the point where the particles melt. Instead, the powder is compressed and heated to the point where micro-bonds are formed between particles. This may include a bond between the powder metal 70 and the first member 22. Once the rod 10k is installed, the micro-bonds may be subjected to fatigue loading, which leads to particle separation over time. Thus, the overall stiffness of the rod 10k may correspondingly vary over time.

FIG. 14 shows an alternative embodiment of rod 10m in which a first member 22 is capped by bioabsorbable plugs 62. As with previous embodiments, the plugs 62 may have a driving feature 64 (e.g., slot, hex, star, cross) that allows the plug 62 to be turned, twisted, pushed, or otherwise inserted into the ends of the rod 10m. The exemplary plugs 62 may be bioabsorbable and dissolve to expose a braided cable 72. The braided cable 72 comprises strands of a biocompatible material such as nylon and is inserted into the interior of the first member 22. The braided cable 72 may be bonded to the first member 22 using a bioabsorbable adhesive. In one embodiment, the braided cable 72 itself may be made from a bioabsorbable material. Thus, over time, the plugs 62 will disintegrate followed by the braided cable 72 and/or the bond between the braided cable 72 and the first member 22. Furthermore, the braided cable 72 substantially fills the first member 22 and permits clamping and bending of the rod 10m to a desired installation shape without kinking the hollow first member 22.

An alternative embodiment of rod 10n is shown in FIG. 15. In this particular embodiment, a first member 74 made from a biocompatible material similar to those described above is sporadically filled with members 76 of a bioabsorbable material. In contrast with previous embodiments, the bioabsorbable members 76 are oriented in a direction other than substantially parallel to the longitudinal axis A. After insertion into the body, these members 76 will dissolve, ultimately leaving a substantially porous first member 74 that has a different stiffness than the originally implanted rod 10n.

The various rod 10 embodiments may have different cross sectional shapes and sizes. For multi-component rods, each of the components may have the same or different shape. By way of example, the embodiment of FIG. 3 illustrates the inner and outer components each having a circular cross section shape. In another embodiment, each of the components has a different shape.

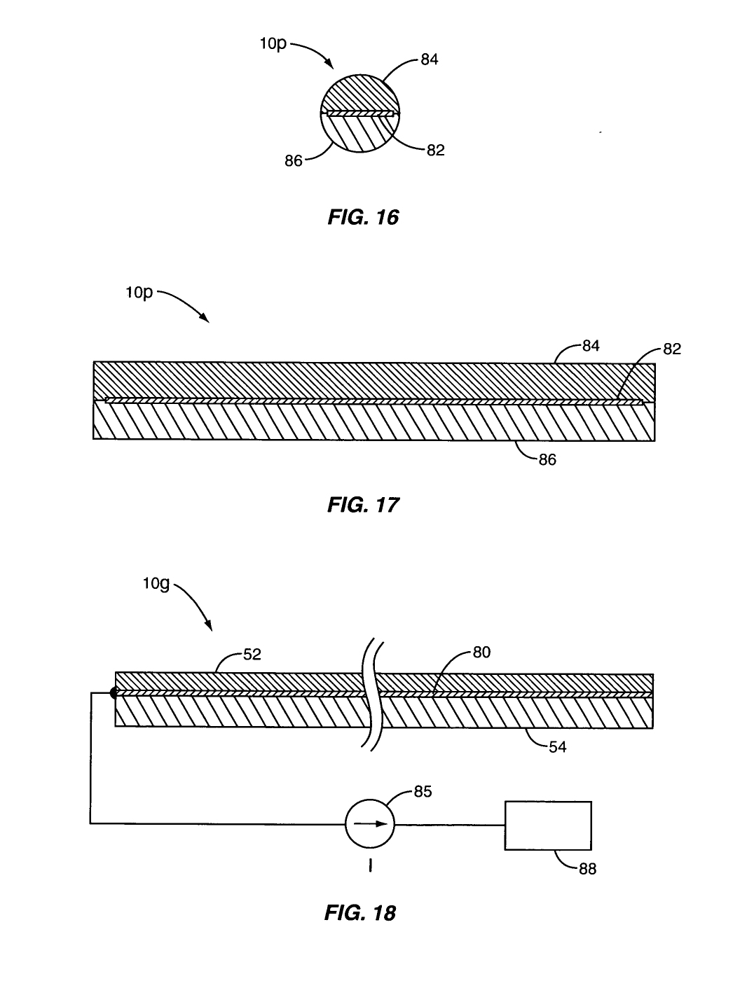

As suggested above, certain embodiments may use metal as a bioabsorbable or biodegradable material. In-vivo corrosion or metal degradation is an electrochemical process. This corrosion can be controlled by altering the electrochemical potential of the metallic implant. In one or more embodiments, two dissimilar metals may be combined to create a galvanic corrosion couple wherein one of the metal members corrodes in a predictable manner. The first metal may be selected from metals that are stable in a biological environment, such as titanium and/or its alloys, niobium and/or its alloys, or tantalum and/or its alloys. The first metal may comprise the substantial portion of the spinal rod. A second metal is that which will undergo corrosion in a biological environment, such as iron and its alloys or magnesium and its alloys. In one embodiment, the second metal is used in combination with the first metal in an arrangement that limits contact between the second metal and the surrounding biological environment to a small area. For example, FIG. 16 illustrates an axial cross section of one embodiment of a rod 10p where a thin sheet 82 of the second metal serves as a thin metallic bond layer between two substantially larger members 84, 86 constructed of the first metal. A longitudinal section view of this same rod 10p is shown in FIG. 17. In the embodiment shown, the thin sheet 82 is disposed substantially within the outer periphery of the outer members 84, 86. That is, the thin sheet 82 is minimally exposed to the surrounding biological environment. Due to the electrochemical nature of the first metal and the relative surface areas of the first and second metals, the second metal will corrode at a slow and relatively predictable rate. The galvanic corrosion rate of the second metal may be enhanced by coating the first metal with a more noble (higher potential) and more electrochemically catalytic metal. Precious metal such as platinum or rhodium and alloys thereof may be used as the coating metals.

Corrosion can also be enhanced or suppressed by controlling the electrochemical potential of the bimetallic composite rod 10p. A current and/or voltage source, such as a neurostimulator, may be used to control this potential. Thus, in one or more embodiments, the rate at which the metal component corrodes (and changes stiffness) may be controlled by connecting the implanted rod 10 to the current or voltage source.

FIG. 18 shows one embodiment incorporating this approach. In this diagram, the rod 10g also illustrated in FIG. 10 is shown in a side section view to demonstrate the exemplary electrical conduction path. Other rod embodiments (e.g., 10, 10a, 10h, 10p, etc . . . ) may be used to implement this technique. In FIG. 18, the first member 52 is bonded to the second member 54 with a biocompatible, bioabsorbable or biodegradable metallic bond layer 80. The bond layer 80 is thin compared to the first member 52 and the second member 54. Furthermore, the bond layer 80 may be more susceptible to corrosion than the adjacent members 52, 54. A current source 85 is coupled at one location to the spinal rod 10g, and to a physically separate electrode 88. The current source 85 and the electrode 88 may be in the immediate vicinity of the structural composite or disposed at a remote location. Suitable materials for the second electrode 88 include, but are not limited to, platinum and/or its alloys, iridium and/or its alloys, or rhodium and/or its alloys.

In one embodiment, the current source 85 is adjusted to supply electrons to the rod 10g and bond layer 80, thereby lowering the electrochemical potential of the rod 10g and inhibiting corrosion of the bond layer 80. In one embodiment, the current source 85 is adjusted to remove electrons from the rod 10g and bond layer 80, thereby raising the electrochemical potential of the rod 10g and enhancing the corrosion rate of the bond layer 80. The current source 85 may be adjustable to either configuration, providing some control over the onset timing and rate of corrosion of the bond layer 80. The current source may be implemented using implantable (e.g., subcutaneous) or external devices. At such time as a clinician desires, the current source 85 may be turned off to initiate spontaneous galvanic corrosion of the bond layer 80 as described above. Consequently, this will decouple the first member 52 and second member 54 and change the structural stiffness of the spinal rod 10g.

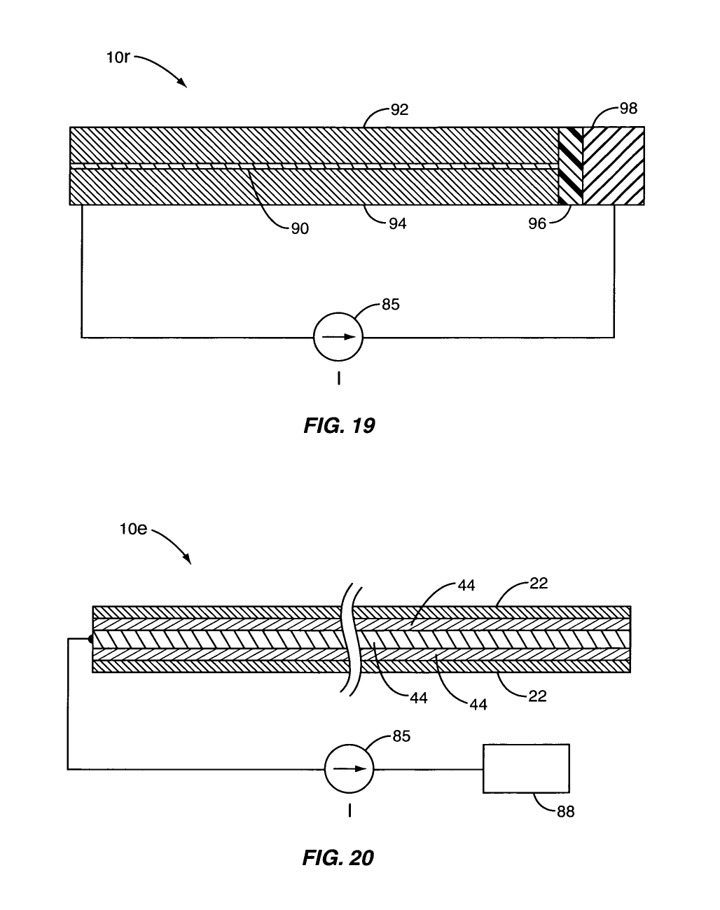

FIG. 19 shows an alternative embodiment incorporating a composite rod 10r. One end of the rod 10r comprises a thin bond layer 90 joining two outer members 92, 94. The opposite end comprises an electrode 98 that is joined to the rod 10r in contrast with the separate electrode 88 shown in FIG. 18. In this embodiment, the electrode 98 is joined to the rod 10r, but electrically insulated from the bond layer 90 and outer members 92, 94 by a non-conductive spacer 96. The non-conductive spacer may be constructed of polymers, resins, ceramics, or other insulating materials. In one embodiment, the current source 85 is adjusted to remove electrons from the outer members 92, 94 and bond layer 90, thereby raising the electrochemical potential of the structural composite and thereby enhancing the corrosion rate of the bond layer 90. In one embodiment, the current source 85 is adjusted to supply electrons to the outer members 92, 94 and bond layer 90, thereby lowering the electrochemical potential of the structural composite and inhibiting corrosion of the bond layer 90. This approach both simplifies implantation of the spinal rod/electrode combination 10r, and allows for a predictable rate of degradation of the second metal.

An alternative embodiment shown in FIG. 20 is similar to the embodiment shown in FIG. 18. In this case, a spinal rod 10e such as that shown in FIG. 8 is depicted. As above, other rod embodiments (e.g., 10c, 10d, 10f, etc . . . ) may be used to implement this technique. In the embodiment depicted in FIG. 20, second members 44 are disposed within an outer first member 22. The second members 44 may be made of a metal that is more susceptible to corrosion than the first member 22. The current source 85 may be connected to preclude corrosion of the second members 44. At such time as the clinician desires, the current source 85 in FIGS. 18, 19, or 20 may be turned off to initiate spontaneous galvanic corrosion of the second members 44. Alternatively, or additionally, the polarity of the current source 85 in FIGS. 18, 19, or 20 can be reversed to further enhance the corrosion rate of members 44. Consequently, the degradation of the second members 44 will change the structural stiffness of the spinal rod 10e.

The present invention may be carried out in other specific ways than those herein set forth without departing from the scope and essential characteristics of the invention. For example, many embodiments described herein use one or more members made from a bioabsorbable material. In general however, certain embodiments, such as the embodiment of rod 10 shown in FIG. 3 may comprise biocompatible materials that are not strictly bioabsorbable. Instead, a bioabsorbable bond similar to that shown in FIG. 6 may be used at interface 30 between non-bioabsorbable first and second members 22, 24. That is, a bioabsorbable bonding interface or other joining mechanism that ultimately disintegrates to separate the first and second members 22, 24 may suffice to achieve the desired time-varying stiffness. The present embodiments are, therefore, to be considered in all respects as illustrative and not restrictive, and all changes coming within the meaning and equivalency range of the appended claims are intended to be embraced therein.

What is claimed is:

1. A spinal rod comprising:
a first member; and
a second member mechanically coupled to the first member through a time-varying interface that degrades after surgical installation.

2. The spinal rod of claim 1 wherein the interface is bioabsorbable and dissolves upon exposure to bodily fluids.

3. The spinal rod of claim 1 wherein the second member is comprised of a bioabsorbable material.

4. The spinal rod of claim 1 wherein the second member is disposed within the first member.

5. The spinal rod of claim 1 wherein the first member and the second member are disposed aside one another.

6. The spinal rod of claim 1 wherein the first member and the second member comprise one or more substantially planar slip planes.

7. The spinal rod of claim 1 wherein the first member and the second member are substantially similar in cross section shape.

8. The spinal rod of claim 1 further comprising one or more bioabsorbable caps to at least temporarily seal the second member from bodily fluids.

9. The spinal rod of claim 1 wherein the second member comprises a sintered powder metal.

10. The spinal rod of claim 1 wherein the second member comprises a braided cable.

11. The spinal rod of claim 1 further comprising an electrode that is electrically insulated from the first and second members.

12. A spinal rod comprising:
a first member; and
a second member;
the first member and the second member coupled to create a first rod stiffness prior to surgical installation, the rod stiffness changing to a second rod stiffness after surgical installation.

13. The spinal rod of claim 12 wherein a cross sectional area of the spinal rod changes after the surgical installation.

14. The spinal rod of claim 12 further comprising a bioabsorbable interface between the first member and the second member.

15. The spinal rod of claim 12 wherein the second member is comprised of a bioabsorbable material.

16. The spinal rod of claim 12 wherein the second member is disposed within the first member.

17. The spinal rod of claim 12 wherein the first member and the second member are disposed aside one another.

18. The spinal rod of claim 12 wherein the first member and the second member comprise one or more substantially planar slip planes.

19. The spinal rod of claim 12 wherein the first member and the second member are substantially similar in cross section shape.

20. The spinal rod of claim 12 further comprising one or more bioabsorbable caps to at least temporarily seal the second member from bodily fluids once the spinal rod is installed.

21. The spinal rod of claim 12 wherein the second member comprises a sintered powder metal.

22. The spinal rod of claim 12 wherein the second member comprises a braided cable.

23. The spinal rod of claim 12 further comprising an electrode that electrically insulated from the first and second members.

24. A spinal rod comprising:
a first member having a tubular shape with a hollow interior with open first and second ends;
a second member positioned within the interior space between the first and second ends; and
end pieces positioned at first and second ends, the end pieces sized to enclose the second member within the hollow interior.

25. The spinal rod of claim 24 the first member, second member and end pieces being constructed from different materials.

26. The spinal rod of claim 24 further comprising an interface that connects the first and second members.

27. The spinal rod of claim 24 wherein the first and second members have different cross sectional shapes.

28. The spinal rod of claim 24 further comprising second end pieces positioned within the hollow interior between the second member and the end pieces.

29. The spinal rod of claim 24 further comprising a third member positioned within the first member.

30. The spinal rod of claim 24 further comprising a notch positioned within the first member and extending along the hollow interior.

31. The spinal rod of claim 30, wherein the notch has a spiral configuration.

32. The spinal rod of claim 24 further comprising a notch extending along a longitudinal length of the second member.

33. A method of using a spinal rod to support a vertebral member, the method comprising the steps of:
connecting a spinal rod to one or more vertebral members;
causing the rod to apply a first mechanical force to the one or more vertebral members;
causing bodily fluids to contact a section of the spinal rod thereby changing a mechanical property of the spinal rod; and
after changing the mechanical property, causing the rod to apply a second mechanical force to the one or more vertebral members, the second mechanical force being different than the first mechanical force.

34. The method of claim 33 wherein the mechanical property of the spinal rod is changed mechanically a predetermined period of time after the step of connecting the spinal rod to the one or more vertebral members.

35. The method of claim 33 wherein the step of changing the mechanical property of the spinal rod comprises dissolving a section of the spinal rod.

36. The method of claim 33 further comprising positioning caps within the spinal rod to control the timing of changing the mechanical property.

37. The method of claim 33 further comprising applying an electrical current to the spinal rod to control the timing of changing the mechanical property.

38. The method of claim 37 wherein applying an electrical current to the spinal rod comprises inducing a current between the spinal rod and an electrode.

39. A method of using a spinal rod to support a vertebral member, the method comprising the steps of:
connecting a spinal rod to one or more vertebral members;
causing the rod to apply a first mechanical force to the one or more vertebral members;
controllably inhibiting the degradation of a bioabsorbable element in the spinal rod;
thereafter changing a mechanical property of the spinal rod thereby causing the rod to apply a second mechanical force to the one or more vertebral members, the second mechanical force being different than the first mechanical force.

40. The method of claim 39 wherein the second mechanical force is less than the first mechanical force.

41. The method of claim 39 further comprising causing bodily fluids to contact the bioabsorbable element.

42. The method of claim 41 wherein controllably inhibiting the degradation of a bioabsorbable element in the spinal rod comprises attaching a fluid barrier to the spinal rod to prevent contact between the bodily fluids and the bioabsorbable member.

43. The method of claim 39 wherein controllably inhibiting the degradation of a bioabsorbable element in the spinal rod comprises applying an electrical current to the bioabsorbable element.

44. The method of claim 43 wherein applying an electrical current to the spinal rod comprises inducing a current between the spinal rod and an electrode.
